(19) United States
(12) Patent Application Publication
(10) Pub. No.: US 2011/0243927 A1
(43) Pub. Date: Oct. 6, 2011

(54) METHODS OF INHIBITING TUMOR GROWTH USING BETA 5 INTEGRIN ANTAGONISTS

(75) Inventor: Guohua Pan, Oakville (CA)

(73) Assignee: University Health Network

(21) Appl. No.: 12/674,300

(22) PCT Filed: Aug. 21, 2008
(86) PCT No.: PCT/CA2008/001485

§ 371 Date: Jun. 7, 2011

Related U.S. Application Data
(60) Provisional application No. 60/966,125, filed on Aug. 24, 2007.
(60) Provisional application No. 61/072,451, filed on Mar. 31, 2008.

(51) Int. Cl. A61K 39/395; A61K 31/713; A61K 38/02; A61P 35/00

(52) U.S. Cl. 424/133.1; 424/130.1; 514/44 A; 514/19.3

(57) ABSTRACT

The present invention relates to methods for treating cancer by administering to a mammalian subject a therapeutically effective amount of a selective β₅ integrin antagonist. The present invention also relates to methods of treating a human epidermal growth factor receptor 2 (HER-2) positive tumor by administering to a mammalian subject a therapeutically effective amount of a β₅ integrin antagonist and a HER-2 antagonist and further relates to compositions of the aforementioned antagonists. The β₅ integrin antagonist can directly inhibit tumor growth by directly inhibiting the activity or the expression of β₅ integrin in a β₅ integrin-expressing tumor cell.

### RELATED APPLICATION(S)

This application claims the benefit of U.S. Provisional Application No. 61/072,451, filed on Mar. 31, 2008 and U.S. Provisional Application No. 60/966,125, filed on Aug. 24, 2007. The entire teachings of the above applications are incorporated herein by reference.

### BACKGROUND OF THE INVENTION

Integrins are a family of cellular receptors that bind extracellular matrix proteins and regulate cell adhesion events. Integrins have been implicated in the aberrant regulation of tumor growth, survival and metastasis.

Integrins are heterodimers comprised of α and β subunits. Numerous α and β subunits and specific heterodimers have been described. For example, the αᵥ subunit chain associates with β₁, β₃, β₅, β₆ and β₈ integrin subunits to form integrin heterodimers. Generally, αᵥ subunit-containing integrins bind ligands having an arginine-glycine-aspartic acid (RGD) tripeptide sequence.

The β₅ integrin associates with αᵥ to form an αᵥβ₅ heterodimer which binds the matrix glycoprotein vitronectin and mediates cell adhesion to and migration on vitronectin. Although the precise role of β₅ integrin in normal physiology is not yet clear, β₅ integrin is preferentially expressed on cancer cells, as opposed to normal cells (see, e.g., Ramaswamy H. and Hemler M. E. EMBO 9(5):1561-1568; Pasqualini et al. J. Cell. Sci. 105:101-111, 1993). In some cell lines, β₅ integrin expression levels exceed that of αᵥ (Pasqualini et al. J. Cell. Sci. 105:101-111, 1993).

Mice homozygous for a null mutation of the β₅ integrin subunit gene exhibit normal development, growth, reproduction and wound healing, although keratinocytes from the β₅ knockout mice have impaired ability to migrate on and adhere to vitronectin. (Huang X. el al., Mol. Cell. Biol. 20(3):755-759, 2000). In contrast, mice containing a null mutation for the αᵥ integrin subunit gene develop intracerebral hemorrhage and die shortly after birth due to abnormal vascular morphogenesis of the brain. The aforementioned studies suggest that the β₅ integrin subunit chain has a role independent of or different from the the αᵥ subunit. Alternatively, β₅ integrin may have binding partners different than the αᵥ subunit.

Both αᵥβ₅ and αᵥβ₃ have been reported to mediate angiogenesis. (See, e.g., Brooks et al. Science 264:569-571 (1994) and WO 97/45447). WO 97/45447 reports that agents that inhibit binding of αᵥβ₅ to vitronectin, such as the anti-αᵥβ₅ antibody P1F6, can be used to inhibit angiogenesis in certain experimental models. This inhibition of tumor angiogenesis is shown in WO 97/45447 to indirectly reduce tumor growth by starving the tumor of nutrients, thereby causing tumor cell necrosis. However, WO 97/45447 does not suggest or demonstrate that agents that bind αᵥβ₅ (e.g., the antibody P1F6) can be used to directly induce cancer cell death. Furthermore, mice that are homozygous null for β₃ integrin, or for both β₃ and β₅ integrins, are reported to have enhanced pathological angiogenesis and tumor growth. These studies call into question the reported role of β₃ and β₅ integrins in angiogenesis. To date, anti-angiogenic therapeutics such as, for example, endostatin have demonstrated little efficacy with respect to tumor regression and patient survival in clinical trials (see e.g., Kulke M. H. et al. J. Clin. Oncol., 24(22):3555-3561, 2006, Hansma A. H. G. et al., Ann. Oncol., 16:1695-1701). Anti-angiogenic therapies have been used successfully as adjuvant treatments with other anti-tumor therapies (e.g., chemotherapy). Thus, anti-angiogenic treatments alone are not sufficient to inhibit tumor growth or progression.

Laug (U.S. Pat. No. 6,521,593) discloses the results of studies of growth of human brain tumor cell lines that expressed αᵥβ₃ and αᵥβ₅. The studies of Laug revealed that selective antagonists of αᵥβ₅, including antibody P1F6, had minimal or no effect on tumor cell adhesion to or migration on vitronectin. In contrast, a non-selective cyclic RGD pentapeptide that inhibited both α_{vβ}₅ and αᵥβ₃ integrins inhibited adhesion and migration, and induced cancer cell death. Id. Thus, the experimental evidence of Laug shows that agents that selectively bind αᵥβ₅ or β₅ integrin were not known to have direct effects on the survival or proliferation of cancer cells.

### SUMMARY OF THE INVENTION

The present invention relates to a method for treating a β₅ integrin positive cancer in a mammalian subject. In the method of the invention, a therapeutically effective amount of a β₅ integrin antagonist is administered to the mammalian subject. In one aspect, the β₅ integrin positive cancer is breast, colon, lung, brain, prostate or ovarian cancer. In another aspect, the β₅ integrin antagonist directly inhibits growth of the tumor by inducing the apoptosis of tumor cells or inhibiting the growth of the tumor cells. In another aspect, the β₅ integrin subunit antagonist inhibits the expression or the activity of the β₅ integrin. The β₅ integrin antagonist can be administered with one or more other therapies. In some embodiments, the β₅ integrin antagonist is administered with a HER-2 antagonist.

The invention also relates to a method for directly inhibiting the growth of a tumor that expresses a β₅ integrin. The method comprises administering to a patient with the tumor a therapeutically effective amount of the β₅ integrin antagonist (for example, with the proviso that that the β₅ integrin antagonist does not bind a αᵥβ₃ heterodimer). In one aspect, the β₅ integrin antagonist directly inhibits growth of the tumor by inducing the apoptosis of the tumor cells or inhibiting the growth of the tumor cells. In another aspect, the β₅ integrin antagonist inhibits the expression or the activity of the β₅ integrin.

The present invention also relates to a method of treating a β₅ integrin positive metastatic tumor by administering to a mammalian subject a therapeutically effective amount of a β₅ integrin antagonist. In one aspect, the β₅ integrin positive metastatic tumor is a breast, lung, colon, prostate, brain or ovary tumor.

The invention further relates to a method of treating a human epidermal growth factor receptor-2 (HER-2) positive tumor by administering to a mammalian subject a therapeutically effective amount of a β₅ integrin antagonist and a therapeutically effective amount of a HER-2 antagonist. In one aspect, the HER-2 positive tumor is a breast cancer tumor. In a particular aspect, the breast cancer tumor also expresses a β₅ integrin. In a further aspect, the breast cancer tumor is HER-2 positive, β₅ integrin positive and estrogen receptor (ER) negative. In some embodiments, a synergistically effective amount of a β₅ integrin antagonist (e.g., anti-β5 antibody) and a HER-2 antagonist (e.g., trastuzumab)are administered.

The invention also relates to a method of treating a luminal A subtype breast cancer or tumor comprising administering to a mammalian subject a therapeutically effective amount of a β₅ integrin antagonist. In one aspect, the method further comprises administering a therapy used for the treatment of a luminal A subtype of breast cancer, such as radiation therapy, chemotherapy, aromatase inhibitors (e.g., anastrozole, exemestane, letrozole) and/or estrogen receptor modulators (e.g., tamoxifen, raloxifene, toremifene).

The invention further relates to a composition comprising a HER-2 receptor antagonist and a β₅ integrin subunit antagonist. In one aspect, the HER-2 receptor antagonist comprising the composition is trastuzumab. In another aspect, the β₅ integrin antagonist comprising the composition is an antibody which selectively binds the β₅ integrin subunit. In some embodiments, the composition comprises a synergistically effective amount of a β₅ integrin antagonist (e.g., anti-β5 antibody) and a HER-2 antagonist (e.g., trastuzumab).

The present invention also relates to method for identifying a candidate for an anti-cancer therapy using a β₅ integrin antagonist comprising providing a tumor sample obtained from a subject and assessing expression of a β₅ integrin in the tumor sample, where expression of β₅ integrin by the tumor or increased expression of β₅ integrin by the tumor relative to a suitable control, indicates that the subject is a candidate for an anti-cancer therapy using a β₅ integrin antagonist.

In contrast with anti-angiogenic therapies, which indirectly inhibit tumor growth and have not produced hoped for benefits in clinical trials to determine efficacy in tumor killing, antagonism of the β₅ integrin subunit in the present invention provides a mechanism by which to treat cancer that involves the direct inhibition of tumor growth. Inhibition of β₅ integrin subunit expression and/or activity using a β₅ integrin subunit antagonist can directly inhibit the proliferation and/or induce the death of β5 integrin subunit-expressing tumor cells. This direct inhibition of tumor growth using β₅ integrin subunit antagonists can be advantageous as compared to anti-angiogenic therapies with respect to efficacy and/or adjuvant therapy requirements.

### BRIEF DESCRIPTION OF THE DRAWINGS

FIG. 1 is a photograph of a western blot illustrating β₅ integrin subunit expression in cancer cells originating from breast, lung, colon and brain tissues.

FIG. 2A is a graph depicting small interfering RNA (siRNA) depletion of β₅ integrin subunit expression in MDA MB-468 breast cancer cells.

FIG. 2B illustrates β₅ integrin siRNA inhibition of the growth of multiple cancer cells. The data represents the percentage of viable cells after 5 days of culture.

FIG. 3 is a series of fluorescence histograms illustrating flow cytometry analysis of breast cancer cells (MCF7 and MDA-MB-468) or normal breast cells (184A1) treated with β₅ integrin siRNA. β₅ integrin siRNA treatment caused apoptosis of the cancer cells (indicated by increased <G1 population in cancer, but not normal cell populations).

FIG. 4 is a graph illustrating the inhibition of MCF7 breast cancer cell growth by an anti-β₅ integrin antibody (KN52) as assessed by an alamarBlue™assay.

FIGS. 5A-5D are graphs illustrating the inhibition of the growth of lung cancer cells (A549) by several anti-β₅ integrin antibodies.

FIG. 6 is the β₅ integrin cDNA sequence (Genbank Accession No. NM002213).

FIG. 7 is the β₅ integrin protein sequence (Genbank Accession No. NP002204).

### DETAILED DESCRIPTION OF THE INVENTION

### Definitions

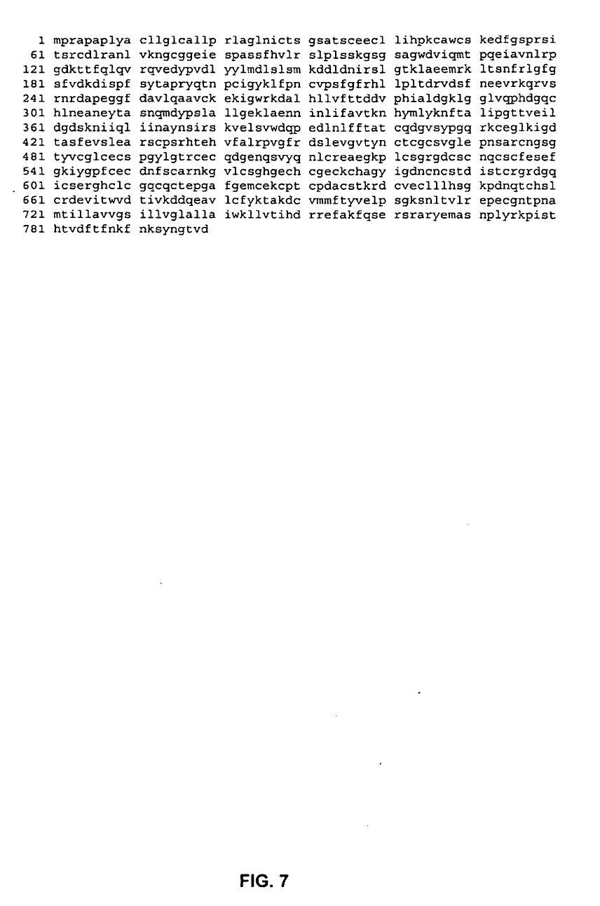

As used herein, “β₅ integrin“, “β₅ integrin subunit” or “β₅ integrin subunit chain” refers to a naturally occurring or endogenous β₅ integrin (e.g., mammalian, human) protein and to proteins having an amino acid sequence which is the same as that of naturally occurring or endogenous β₅ integrin protein (e.g., recombinant proteins, synthetic proteins). Accordingly, “β₅ integrin”, “β₅ integrin subunit” or “β₅ integrin subunit chain” includes polymorphic or allelic variants and other isoforms of a β₅ integrin (e.g., mammalian, human) produced by, e.g., alternative splicing or other cellular processes, that occur naturally in mammals (e.g., humans, non-human primates). Preferably, the β₅ integrin is a human protein that has the amino acid sequence of SEQ ID NO: 2. (See, Genbank Accession No. NP002204 and FIG. 7).

As defined herein, a “β₅ integrin antagonist” is an agent (e.g., molecule, protein, polypeptide, antibody, compound) which specifically and selectively binds a β₅ integrin and inhibits one or more activities of a β₅ integrin and/or a β₅ integrin heterodimer; or an agent that inhibits (e.g., reduces, prevents) the expression of a β₅ integrin subunit gene and/or protein. A β₅ integrin antagonist can bind a β₅ integrin subunit alone or in a heterodimeric integrin complex (e.g., a αᵥβ₅ complex). A β₅ integrin antagonist can, for example, inhibit binding of a ligand (e.g., vitronectin) to β₅ integrin or a β₅ integrin heterodimer, such as αᵥβ₅, by, for instance, blocking the ligand-binding site (e.g., RGD recognition site). A β₅ integrin antagonist can inhibit the activity of a β₅ integrin in response to ligand binding, for example, the binding of cytosolic proteins (e.g., annexin V (see Cardó-Vila et al., Mol. Cell. 11:1151-1162, 2003)) or cytosolic signaling of the receptor (e.g., outside-in signaling or inside-out signaling)). A β₅ integrin antagonist that inhibits the expression and/or activity of a β₅ integrin and can be, for example, a natural or synthetic nucleic acid or nucleic acid analog, antisense molecule, small interfering RNA (siRNA), protein, peptide, antibody, chemical compound or the like. As defined herein, a β₅ integrin antagonist selectively binds or inhibits expression of the β₅ integrin subunit chain and, therefore, does not bind other β subunits (e.g., β₁, β₃, β₆, β₈) or other β subunit-containing heterodimer complexes (e.g., αᵥβ₃, αᵥβ₁) under physiological conditions.

As used herein, the term “peptide”, refers to a compound consisting of from about 2 to about 100 amino acid residues wherein the amino group of one amino acid is linked to the carboxyl group of another amino acid by a peptide bond. Such peptides are typically less than about 100 amino acid residues in length and preferably are about 10, about 20, about 30, about 40 or about 50 residues.

As used herein, the term “peptidomimetic”, refers to molecules which are not polypeptides, but which mimic aspects of their structures. Peptidomimetic antagonists can be prepared by conventional chemical methods (see e.g., Damewood J. R. “Peptide Mimetic Design with the Aid of Computational Chemistry” in Reviews in Computational Biology, 2007, Vol. 9, pp. 1-80, John Wiley and Sons, Inc., New York, 1996; Kazmierski W. K., “Methods of Molecular Medicine: Peptidomimetic Protocols,” Humana Press, New Jersey, 1999).

As defined herein, a “therapy” is the administration of a particular therapeutic or prophalytic agent to a subject (e.g., mammalian, human).

As defined herein a “treatment regimen” is a regimen in which one or more therapeutic or prophalytic agents are administered to a mammalian subject at a particular dose (e.g., level, amount, mass) and on a particular schedule or at particular intervals (e.g., minutes, days, weeks, months).

As defined herein, “direct inhibition of tumor growth” refers to inhibited tumor growth (e.g., reduced tumor cell proliferation, tumor cell death) caused by the interaction of a therapeutic agent with a target in or on a tumor cell. Thus, a β₅ integrin antagonist can directly inhibit tumor growth by binding a β₅ integrin or β₅ integrin-containing heterodimer expressed by the cells of the tumor and inhibiting the activity of the β₅ integrin or β₅ integrin-containing heterodimer, for example. In addition, a β₅ integrin antagonist can directly inhibit tumor growth by inhibiting expression (e.g., decreasing nucleic acid (e.g., RNA) and/or protein) of a β₅ integrin in the cells of the tumor.

As defined herein, a “therapeutically effective amount” is an amount sufficient to achieve the desired therapeutic or prophylactic effect under the conditions of administration, such as an amount sufficient to inhibit (i.e., reduce, prevent) tumor cell growth (proliferation, size) and/or tumor progression (invasion, metastasis) for a particular cancer. The effectiveness of a therapy (e.g., the reduction/elimination of a tumor and/or prevention tumor growth) can be determined by suitable methods (e.g., in situ immunohistochemistry, imaging (MRI, NMR), ³H-thymidine incorporation).

As defined herein, an “anti-tumor effective amount” is an amount sufficient to directly inhibit tumor cell growth (e.g., proliferation) or survival.

As defined herein, an “anti-angiogenic effective amount” is an amount sufficient to inhibit angiogenesis.

As defined herein, a “synergistic amount” or “synergistically effective amount” is an amount of two or more agents that produces greater than expected additive effect based on the mass-action law. A synergistic amount or synergistically effective amount has as combination index of less than one (CI<1). Preferably, the synergistic amount or synergistically effective amount has a CI of ≦0.85 (e.g., 0.7-0.85), ≦0.7 (e.g., 0.3-0.7), ≦0.3 (e.g., 0.1-0.3) or ≦0.1. The CI, method for calculating CI and plots useful for visualizing CI and synergistic, additive and antagonistic combinations are described in Chou, T-C., Theoretical Basis, Experimental Design, and Computerized Simulation of Synergism and Antagonism in Drug Combination Studies, Pharmacological Reviews 58(3):621-681 (2006). The skilled addressee is directed, in particular, to section II regarding methods for calculating CI and plots useful for visualizing CI and synergistic, additive and antagonistic combinations. The entire teachings of Chou, T-C., Theoretical Basis, Experimental Design, and Computerized Simulation of Synergism and Antagonism in Drug Combination Studies, Pharmacological Reviews 58(3):621-681 (2006), including the portions specifically referred to herein, are incorporated herein by reference.

As described herein, the inventors have discovered that antagonists of β₅ integrin can directly inhibit proliferation (e.g., by inducing apoptosis, inhibiting cell growth) of β₅ integrin subunit-expressing tumor cells. Thus, antagonists of β₅ integrin can inhibit tumor growth and/or progression (e.g, in cancer patients). Accordingly, the invention provides a method for the targeted therapy of a cancer (e.g., a tumor) that expresses a β₅ integrin. The inventors have also discovered that β₅ integrin is expressed in particular cancers and/or expressed at higher levels, relative to normal cells or tissue, in some particular cancers, such as breast cancer, particularly HER-2 positive/estrogen receptor negative and luminal A subtypes of breast cancer. HER-2 has been found to promote cancer growth, and HER-2 positive tumors are generally more aggressive and less responsive to certain therapies (e.g., hormone therapy), than other types of breast tumors. Accordingly, the invention also provides a method for the targeted therapy of a cancer (e.g., breast cancer) that expresses HER-2 and expresses a β₅ integrin subunit chain and, further, provides for a composition comprising a HER-2 antagonist and a β₅ integrin antagonist. As shown herein, a HER-2 antagonist and a β5 integrin antagonist can be coadministered to produce a synergistic effect, thereby providing superior therapy, for example, for cancer or tumors that express or over-express HER-2 and β₅ integrin subunit chain. The invention also provides a method of treating luminal A subtype breast cancer by administering a β₅ integrin antagonist alone or in combination with another therapy for luminal-A subtype breast cancer.

The inhibition of the expression or activity of a β₅ integrin provides an effective and selective mechanism by which to treat tumors which express a β₅ integrin. Thus, one aspect the present invention relates to a method for treating cancer in a mammalian subject comprising administering to the subject a therapeutically effective amount of a β₅ integrin antagonist.

### β₅ Integrin Antagonists

The β₅ integrin antagonist can be an antibody or antigen-binding fragment thereof which selectively binds a β₅ integrin protein, for example, alone or in a β₅ heterodimer. The term “antibody” is intended to encompass all types of polyclonal and monoclonal antibodies (e.g., human, chimeric, humanized, primatized, veneered, single chain, domain antibodies (dAbs)) and antigen-binding fragments of antibodies (e.g., Fv, Fc, Fd, Fab, Fab′, F(ab′), dAb). (See e.g., Harlow et al., Antibodies A Laboratory Manual, Cold Spring Harbor Laboratory, 1988). In a particular embodiment, the β₅ integrin-specific antibody is a human antibody or humanized antibody. β₅ integrin-specific antibodies can also be directly or indirectly linked to a cytotoxic agent.

Several antibodies which selectively bind β₅ integrin (e.g., KN52, B5-IVF2) or a αᵥβ₅ heterodimer (e.g., P5H9, P1F6) have been produced and are commercially available (e.g., from eBiosciences, R & D Systems, Abcam PLC, USBIO, Sigma, Everestbiotech, Novusbio, SCBT, Abnova). Other antibodies or antibody fragments which selectively bind to and inhibit the activity of a β₅ integrin and/or a β₅ integrin-containing heterodimer can also be produced, constructed, engineered and/or isolated by conventional methods or other suitable techniques. For example, antibodies which are specific for a β₅ integrin and/or a β₅ integrin-containing complex can be raised against an appropriate immunogen, such as a recombinant mammalian (e.g., human) β₅ integrin subunit chain or portion thereof (including synthetic molecules, e.g., synthetic peptides). A variety of methods have been described (see e.g., Kohler et al., Nature, 256: 495-497 (1975) and Eur. J. Immunol. 6: 511-519 (1976); Milstein et al., Nature 266: 550-552 (1977); Koprowski et al., U.S. Pat. No. 4,172,124; Harlow, E. and D. Lane, 1988, Antibodies: A Laboratory Manual, (Cold Spring Harbor Laboratory: Cold Spring Harbor, N.Y.); Current Protocols In Molecular Biology, Vol. 2 (Supplement 27, Summer '94), Ausubel, F. M. et al., Eds., (John Wiley & Sons: New York, N.Y.), Chapter 11, (1991)). Antibodies can also be raised by immunizing a suitable host (e.g., mouse) with cells that express β₅ integrin (e.g., cancer cells/cell lines) or cells engineered to express β₅ integrin (e.g., transfected cells). (See e.g., Chuntharapai et al., J. Immunol., 152:1783-1789 (1994); Chuntharapai et al. U.S. Pat. No. 5,440,021). For the production of monoclonal antibodies, a hybridoma can be produced by fusing a suitable immortal cell line (e.g., a myeloma cell line such as SP2/0 or P3X63Ag8.653) with antibody producing cells. The antibody producing cells can be obtained from the peripheral blood, or preferably, the spleen or lymph nodes, of humans or other suitable animals immunized with the antigen of interest. The fused cells (hybridomas) can be isolated using selective culture conditions, and cloned by limited dilution. Cells which produce antibodies with the desired specificity can be selected by a suitable assay (e.g., ELISA).

Antibody fragments can be produced by enzymatic cleavage or by recombinant techniques. For example, papain or pepsin cleavage can generate Fab or F(ab′)₂ fragments, respectively. Other proteases with the requisite substrate specificity can also be used to generate Fab or F(ab′)₂ fragments. Antibodies can also be produced in a variety of truncated forms using antibody genes in which one or more stop codons has been introduced upstream of the natural stop site. For example, a chimeric gene encoding a F(ab′)₂ heavy chain portion can be designed to include DNA sequences encoding the CH₁ domain and hinge region of the heavy chain. Single chain antibodies, and human, chimeric, humanized or primatized (CDR-grafted), or veneered antibodies, as well as chimeric, CDR-grafted or veneered single chain antibodies, comprising portions derived from different species, and the like are also encompassed by the present invention and the term “antibody”. The various portions of these antibodies can be joined together chemically by conventional techniques, or can be prepared as a contiguous protein using genetic engineering techniques. For example, nucleic acids encoding a chimeric or humanized chain can be expressed to produce a contiguous protein. See, e.g., Cabilly et al., U.S. Pat. No. 4,816,567; Cabilly et al., European Patent No.

0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., European Patent No. 0 451 216 B1; and Padlan, E. A. et al., EP 0 519 596 A1. See also, Newman, R. et al., BioTechnology, 10: 1455-1460 (1992), regarding primatized antibody, and Ladner et al., U.S. Pat. No. 4,946,778 and Bird, R. E. et al., Science, 242: 423-426 (1988)) regarding single chain antibodies.

Humanized antibodies can be produced using synthetic or recombinant DNA technology using standard methods or other suitable techniques. Nucleic acid (e.g., cDNA) sequences coding for humanized variable regions can also be constructed using PCR mutagenesis methods to alter DNA sequences encoding a human or humanized chain, such as a DNA template from a previously humanized variable region (see e.g., Kamman; M., et al., Nucl. Acids Res., 17: 5404 (1989)); Sato, K., et al., Cancer Research, 53: 851-856 (1993); Daugherty, B. L. et al., Nucleic Acids Res., 19(9): 2471-2476 (1991); and Lewis, A. P. and J. S. Crowe, Gene, 101: 297-302 (1991)). Using these or other suitable methods, variants can also be readily produced. In one embodiment, cloned variable regions (e.g., dAbs) can be mutated, and sequences encoding variants with the desired specificity can be selected (e.g., from a phage library; see e.g., Krebber et al., U.S. Pat. No. 5,514,548; Hoogenboom et al., WO 93/06213, published Apr. 1, 1993).

Other suitable methods of producing or isolating antibodies of the requisite specificity can be used, including, for example, methods which select a recombinant antibody or antibody-binding fragment (e.g., dAbs) from a library (e.g., a phage display library), or which rely upon immunization of transgenic animals (e.g., mice). Transgenic animals capable of producing a repertoire of human antibodies are well-known in the art (e.g., Xenomous® (Abgenix, Fremont, Calif.)) and can be produced using suitable methods (see e.g., Jakobovits et al., Proc. Natl. Acad. Sci. USA, 90: 2551-2555 (1993); Jakobovits et al., Nature, 362: 255-258 (1993); Lonberg et al., U.S. Pat. No. 5,545,806; Surani et al., U.S. Pat. No. 5,545,807; Lonberg et al., WO 97/13852).

A β₅ integrin antagonist can be a peptide (e.g., synthetic, recombinant, fusion or derivatized) which specifically binds to and inhibits (reduces, prevents, decreases) the activity of the β₅ integrin or a β₅ integrin-containing heterodimer. The peptide can be linear, branched or cyclic, e.g., a peptide having a heteroatom ring structure that includes several amide bonds. In a particular embodiment, the peptide is a cyclic peptide.

Peptides, including cyclic peptides, that are selective for binding to a particular domain (e.g., unique domain) of a β₅ integrin or a β₅ integrin-containing heterodimer can be produced. A peptide can be, for example, derived or removed from a native protein by enzymatic or chemical cleavage, or can be synthesized by suitable methods, for example, solid phase peptide synthesis (e.g., Merrifield-type synthesis) (see, e.g., Bodanszky et al. “Peptide Synthesis,” John Wiley & Sons, Second Edition, 1976). Peptides that are β₅ integrin antagonists can also be produced, for example, using recombinant DNA methodologies or other suitable methods (see, e.g., Sambrook J. and Russell D. W., Molecular Cloning: A Laboratory Manual, 3^{rd} Edition, Cold Spring Harbor Laboratory Press, Cold Spring Harbor, N.Y., 2001). β₅ integrin antagonists can also be fusion peptides fused, for example to a carrier protein (e.g., myc, his, glutathione sulfhydryl transferase) and/or tagged (e.g., radiolabeled, fluorescently labeled).

A peptide can comprise any suitable L-and/or D-amino acid, for example, common α-amino acids (e.g., alanine, glycine, valine), non-α-amino acids (e.g., β-alanine, 4-aminobutyric acid, 6-aminocaproic acid, sarcosine, statine), and unusual amino acids (e.g., citrulline, homocitruline, homoserine, norleucine, norvaline, ornithine). The amino, carboxyl and/or other functional groups on a peptide can be free (e.g., unmodified) or protected with a suitable protecting group. Suitable protecting groups for amino and carboxyl groups, and methods for adding or removing protecting groups are known in the art and are disclosed in, for example, Green and Wuts, “Protecting Groups in Organic Synthesis”, John Wiley and Sons, 1991. The functional groups of a peptide can also be derivatized (e.g., alkylated) using art-known methods.

Peptides can be synthesized and assembled into libraries comprising a few to many discrete molecular species. Such libraries can be prepared using methods of combinatorial chemistry, and can be screened using any suitable method to determine if the library comprises peptides with a desired biological activity. Such peptide antagonists can then be isolated using suitable methods.

In particular, a peptide antagonist containing a RGD sequence or a sequence similar to the RGD sequence can be designed that is selective for inhibition of αᵥβ₅ (see e.g., WO 97/45447, incorporated herein by reference) or other β₅ integrin-containing heterodimers. The polypeptide can comprise modifications (e.g., amino acid linkers, acylation, acetylation, amidation, methylation, terminal modifiers (e.g., cyclizing modifications)), if desired. The polypeptide can also contain chemical modifications (e.g., N-methyl-α-amino group substitution). In addition, the peptide antagonist can be an analog of a known and/or naturally-occurring peptide, for example, a peptide analog having conservative amino acid residue substitution(s). These modifications can improve various properties of the peptide (e.g., solubility, binding), including its β₅ integrin antagonist activity.

Peptidomimetics can be prepared that are β₅ integrin antagonists. For example, polysaccharides can be prepared that have the same functional groups as peptides. Peptidomimetics can be designed, for example, by establishing the three dimensional structure of a peptide agent in the environment in which it is bound or will bind to a target molecule. The peptidomimetic comprises at least two components, the binding moiety or moieties and the backbone or supporting structure.

The binding moieties are the chemical atoms or groups which will react or form a complex (e.g., through hydrophobic or ionic interactions) with a target molecule, for example, with the amino acid(s) at or near the ligand binding site. For example, the binding moieties in a peptidomimetic can be the same as those in a peptide or protein antagonist. The binding moieties can be an atom or chemical group which reacts with the receptor in the same or similar manner as the binding moiety in the peptide antagonist. For example, computational chemistry can be used to design peptide mimetics of the vitronectin binding site and/or RGD recognition site of the β₅ integrin subunit ligand-binding domain to inhibit the activity of a αᵥβ₅ heterodimer, for instance. Examples of binding moieties suitable for use in designing a peptidomimetic for a basic amino acid in a peptide include nitrogen containing groups, such as amines, ammoniums, guanidines and amides or phosphoniums. Examples of binding moieties suitable for use in designing a peptidomimetic for an acidic amino acid include, for example, carboxyl, lower alkyl carboxylic acid ester, sulfonic acid, a lower alkyl sulfonic acid ester or a phosphorous acid or ester thereof. The design, production and various examples of β₅ integrin subunit peptide mimetics can be found in WO 97/45447.

The supporting structure is the chemical entity that, when bound to the binding moiety or moieties, provides the three dimensional configuration of the peptidomimetic. The supporting structure can be organic or inorganic. Examples of organic supporting structures include polysaccharides, polymers or oligomers of organic synthetic polymers (such as, polyvinyl alcohol or polylactide). It is preferred that the supporting structure possess substantially the same size and dimensions as the peptide backbone or supporting structure. This can be determined by calculating or measuring the size of the atoms and bonds of the peptide and peptidomimetic. In one embodiment, the nitrogen of the peptide bond can be substituted with oxygen or sulfur, for example, forming a polyester backbone. In another embodiment, the carbonyl can be substituted with a sulfonyl group or sulfinyl group, thereby forming a polyamide (e.g., a polysulfonamide). Reverse amides of the peptide can be made (e.g., substituting one or more-CONH-groups for a-NHCO-group). In yet another embodiment, the peptide backbone can be substituted with a polysilane backbone.

These compounds can be manufactured by known methods. For example, a polyester peptidomimetic can be prepared by substituting a hydroxyl group for the corresponding α-amino group on amino acids, thereby preparing a hydroxyacid and sequentially esterifying the hydroxyacids, optionally blocking the basic and acidic side chains to minimize side reactions. Determining an appropriate chemical synthesis route can generally be readily identified upon determining the chemical structure.

Peptidomimetics can be synthesized and assembled into libraries comprising a few to many discrete molecular species. Such libraries can be prepared using well-known methods of combinatorial chemistry, and can be screened to determine if the library comprises one or more peptidomimetics which have the desired activity. Such peptidomimetic antagonists can then be isolated by suitable methods.

Other β₅ integrin antagonists like, for example, non-peptidic compounds or small molecules, can be found in nature (e.g., identified, isolated, purified) and/or produced (e.g., synthesized). Agents can be tested for β₅ integrin binding specificity in a screen for example, a high-throughput screen of chemical compounds and/or libraries (e.g., chemical, peptide, nucleic acid libraries). Compounds or small molecules can be identified from numerous available libraries of chemical compounds from, for example, the Chemical Repository of the National Cancer Institute, the Molecular Libraries Small Molecules Repository (PubChem) and other libraries that are commercially available. Such libraries or collections of molecules can also be prepared using well-known chemical methods, such as well-known methods of combinatorial chemistry. The libraries can be screed to identify compounds that bind and inhibit β5 integrin. Identified compounds can serve as lead compounds for further diversification using well-known methods of medicinal chemistry. For example, a collection of compounds that are structural variants of the lead can be prepared and screed for β5 integrin binding and/or inhibiting activity. This can result in the development of an structure activity relationship that links the structure of the compounds to biological activity. Compounds that have suitable binding and inhibitory activity can be further developed for in vivo use.

Agents which bind β₅ integrin can be further evaluated for β₅ integrin antagonist activity. A composition comprising a β₅ integrin or a β₅ integrin heterodimer (e.g., αᵥβ₅) can be used in a such a screen or binding assay to detect and/or identify agents that can bind to a β₅ integrin or a β₅ integrin heterodimer. Compositions suitable for use include, for example, cells which naturally express a β5 integrin and/or a β₅ integrin heterodimer (e.g., cancer cell lines A549, A431, BT20, ADAH, MCF-7, J82, HeLa, MIP, HUVEC, HT1080, MG-63, SKMEL, LOX, SKNSH; Pasqualini R. et al, J. Cell Sci. 105:101-111, 1993). Other suitable compositions for use in a binding assay include, for example, membrane preparations (e.g., natural (e.g., plasma) or synthetic membranes) which comprise a β₅ integrin, a β₅ integrin heterodimer or functional fragments thereof.

An agent that binds a β₅ integrin or β₅ integrin-containing heterodimer can be identified in a competitive binding assay, for example, in which the ability of a test agent to inhibit the binding of a reference agent (e.g., a ligand (e.g., vitronectin)) is assessed. The reference agent can be labeled with a suitable label (e.g., radioisotope, epitope label, affinity label (e.g., biotin and avidin or streptavadin), spin label, enzyme, fluorescent group, chemiluminescent group, dye, metal (e.g., gold, silver), magnetic bead) and the amount of labeled reference agent required to saturate the β₅ integrin subunit or a β₅ integrin subunit receptor complex in the assay can be determined. The specificity of the formation of the complex between the β₅ integrin subunit and the test agent can be determined using a suitable control (e.g., unlabeled agent, label alone).

The capacity of a test agent to inhibit formation of a complex between the reference agent and a β₅ integrin or a β₅ integrin heterodimer can be determined as the concentration of test agent required for 50% inhibition (IC₅₀ value) of specific binding of labeled reference agent. Specific binding is preferably defined as the total binding (e.g., total label in complex) minus the non-specific binding. Non-specific binding is preferably defined as the amount of label still detected in complexes formed in the presence of excess unlabeled reference agent. Reference agents suitable for use in the method include molecules and compounds which specifically bind to β₅ integrin or a β₅ integrin heterodimer, e.g., a ligand of a β₅ integrin or a β₅ integrin receptor heterodimer (e.g., vitronectin) or an antibody specific for β₅ integrin (e.g., KN52, eBiosciences; B5-IVF2, Abcam, PLC) or a β₅ integrin heterodimer (e.g., P5H9, R & D Systems).

An agent which binds a β₅ integrin or a β₅ integrin heterodimer can be further studied to assess the ability of that agent to antagonize (reduce, prevent, inhibit) one or more functions of the β₅ integrin subunit or β₅ integrin heterodimer. Functional characteristics of a β₅ integrin include binding activities (e.g., ligand binding), signaling activities (e.g., cell-cell or cell-matrix signaling) and/or an ability to stimulate a cellular response (e.g., outside-in signaling) or stimulate ligand binding (e.g., inside-out signaling). In vitro, β₅ integrin induces angiogenesis, cell migration, cell adhesion and cell proliferation. Thus, assays detecting these β₅ integrin-mediated functions can be used to evaluate the antagonist activity of a test agent (e.g., the ability of a test agent to inhibit one or more functions of β₅ integrin). Exemplary assays include model angiogenesis assays (e.g., ocular angiogenesis, chicken chorioallantoric membrane (CAM), liquid Matrigel, animal xenograft), Transwell migration chamber assays, vitronectin adhesion assays and cell proliferation assays (e.g., BrdU incorporation, ³H-thymidine incorporation). (See e.g., Friedlander M. et al. Science 270:1500-1502, 1995; Klemke R. L., J. Cell. Biol. 131:791-805, 1995; Kerr J. S. et al., Anticancer Res. 19:959-968, 1999).

β₅ integrin antagonists are also agents that inhibit (reduce, decrease, prevent) the expression of a β₅ integrin. Agents (molecules, compounds, nucleic acids, oligonucleotides) which inhibit β₅ integrin subunit gene expression (e.g., transcription, mRNA processing, translation) are effective β₅ integrin antagonists. For example, small interfering ribonucleic acids (siRNAs) and, similarly, short hairpin ribonucleic acids (shRNAs) which are processed into short siRNA-like molecules in a cell, can prevent the expression (translation) of the β₅ integrin subunit chain protein. siRNA molecules can be polynucleotides that are generally about 20 to about 25 nucleotides long and are designed to bind specific RNA sequence (e.g., β₅ integrin mRNA). siRNAs silence gene expression in a sequence-specific manner, binding to a target RNA (e.g., an RNA having the complementary sequence) and causing the RNA to be degraded by endoribonucleases. siRNA molecules able to inhibit the expression of the β₅ integrin subunit gene product can be produced by suitable methods. There are several algorithms that can be used to design siRNA molecules that bind the sequence of a gene of interest (see e.g., Mateeva O. et al. Nucleic Acids Res. 35(8):Epub, 2007; Huesken D. et al., Nat. Biotechnol. 23:995-1001; Jagla B. et al., RNA 11:864-872, 2005; Shabalinea S. A. BMC Bioinformatics 7:65, 2005; Vert J. P. et al. BMC Bioinformatics 7:520, 2006). Expression vectors that can stably express siRNA or shRNA are available. (See e.g., Brummelkamp, T. R., Science 296: 550-553, 2002, Lee, N S, et al., Nature Biotechnol. 20:500-505, 2002; Miyagishi, M., and Taira, K. Nature Biotechnol. 20:497-500, 2002; Paddison, P. J., et al., Genes & Dev. 16:948-958, 2002; Paul, C. P., et al., Nature Biotechnol. 20:505-508; 2002; Sui, G., et al., Proc. Natl. Acad. Sci. USA 99(6):5515-5520, 2002; Yu, J-Y, et al., Proc. Natl. Acad. Sci. USA 99(9):6047-6052, 2002; Elbashir, S M, et al., Nature 411:494-498, 2001.). Stable expression of siRNA/shRNA molecules is advantageous in the treatment of cancer as it enables long-term expression of the molecules, potentially reducing and/or eliminating the need for repeated treatments.

Antisense oligonucleotides (e.g., DNA, riboprobes) can also be used as β₅ integrin antagonists to inhibit β₅ integrin subunit expression. Antisense oligonucleotides are generally short (˜13 to ˜25 nucleotides) single-stranded nucleic acids which specifically hybridize to a target nucleic acid sequence (e.g., mRNA) and induce the degradation of the target nucleic acid (e.g., degradation of the RNA through RNase H-dependent mechanisms) or sterically hinder the progression of splicing or translational machinery. (See e.g., Dias N. and Stein C. A., Mol. Can. Ther. 1:347-355, 2002). There are a number of different types of antisense oligonucleotides that can be used as β₅ integrin antagonists including methylphosphonate oligonucleotides, phosphorothioate oligonucleotides, oligonucleotides having a hydrogen at the 2′-position of ribose replaced by an O-alkyl group (e.g., a methyl), polyamide nucleic acid (PNA), phosphorodiamidate morpholino oligomers (deoxyribose moiety is replaced by a morpholine ring), PN (N3′→P5′ replacement of the oxygen at the 3′ position on ribose by an amine group) and chimeric oligonucleotides (e.g., 2′-O-Methyl/phosphorothioate). Antisense oligonucleotides can be designed to be specific for a β₅ integrin using predictive algorithms. (See e.g., Ding, Y., and Lawrence, C. E., Nucleic Acids Res., 29:1034-1046, 2001; Sczakiel, G., Front. Biosci., 5:D194-D201, 2000; Scherr, M., et al., Nucleic Acids Res., 28:2455-2461, 2000; Patzel, V., et al. Nucleic Acids Res., 27:4328-4334,1999; Chiang, M. Y., et al. J. Biol. Chem., 266:18162-18171,1991; Stull, R. A., et al., Nucleic Acids Res., 20:3501-3508, 1992; Ding, Y., and Lawrence, C. E., Comput. Chem., 23:387-400,1999; Lloyd, B. H., et al., Nucleic Acids Res., 29:3664-3673, 2001; Mir, K. U., and Southern, E. M., Nat. Biotechnol., 17:788-792,1999; Sohail, M., et al., Nucleic Acids Res., 29:2041-2051, 2001; Altman, R. K., et al., J. Comb. Chem., 1:493-508, 1999). The antisense oligonucleotides can be produced by suitable methods; for example, nucleic acid (e.g., DNA, RNA, PNA) synthesis using an automated nucleic acid synthesizer (from, e.g., Applied Biosystems) (see also Martin, P., Helv. Chim. Acta 78:486-504, 1995). Antisense oligonucleotides can also be stably expressed in a cell containing an appropriate expression vector.

Antisense oligonucleotides can be taken up by target cells (e.g., tumor cells) via the process of adsorptive endocytosis. Thus, in the treatment of a subject (e.g., mammalian), antisense β₅ integrin can be delivered to target cells (e.g., tumor cells) by, for example, injection or infusion. For instance, purified oligonucleotides or siRNA/shRNA, can be administered alone or in a formulation with a suitable drug delivery vehicle (e.g., liposomes, cationic polymers, (e.g., poly-L-lylsine PAMAM dendrimers, polyalkylcyanoacrylate nanoparticles and polyethyleneimine) or coupled to a suitable carrier peptide (e.g., homeotic transcription factor, the Antennapedia peptide, Tat protein of HiV-1, E5CA peptide).

### Methods of Therapy

Using the methods of the invention, tumor growth can be inhibited (e.g., directly inhibited) using a β₅ integrin antagonist (e.g., antibodies, siRNA molecules, antisense oligonucleotides, chemical compounds, peptides, peptide mimetics, non-peptidic molecules). This is in contrast to anti-angiogenic treatments which block angiogenic growth factor signals (e.g., endostatin, tumstatin, angiostatin) or the response of endothelial cells in the tumor bed to those signals (e.g., avastatin), and are used as adjunct therapies to anti-tumor therapy.

Accordingly, one aspect of the invention relates to a method for treating a β₅ integrin positive cancer in a mammalian subject comprising administering to the subject a therapeutically effective amount of a β₅ integrin antagonist. In a particular aspect of the method, a β₅ integrin subunit antagonist inhibits tumor growth directly by inducing the death (e.g., apoptosis) of the cells of the tumor or by inhibiting the growth (e.g., proliferation) of the cells of the tumor. Another aspect of the invention relates to a method of treating a β₅ integrin positive metastatic tumor (cancer). The cancer/tumor treated with a β₅ integrin antagonist can be any cancer (e.g., carcinoma, sarcoma, melanoma, fibrosarcoma, neuroblastoma, rabdomyosarcoma, myeloid, endothelial, epithelial, breast, cervical, colon, bladder, skin, prostate, brain) or particular type of tumor (e.g., primary, nodal, metastatic) which expresses β₅ integrin alone, or in a heterodimer (e.g., αᵥβ₅). In a particular embodiment, the cancer and/or tumor treated is breast cancer, lung cancer, colon cancer, prostate cancer, ovarian cancer and/or brain cancer. In more particular embodiments, β₅ integrin positive/luminal A type breast cancer or β₅ integrin positive/HER-2 positive/estrogen receptor negative type breast cancer is treated.

A therapeutically effective amount of the β₅ integrin antagonist is administered in the methods of the invention. In one aspect, an “anti-tumor effective amount” of a β₅ integrin antagonist is administered to a patient in need thereof. For example, agents which directly inhibit tumor growth (e.g., chemotherapeutic agents) are conventionally administered at a particular dosing schedule and level to achieve the most effective therapy (e.g., to best kill tumor cells). Generally, about the maximum tolerated dose is administered during a relatively short treatment period (e.g., one to several days), which is followed by an off-therapy period. In a particular example, the chemotherapeutic cyclophosphamide is administered at a maximum tolerated dose of 150 mg/kg every other day for three doses, with a second cycle given 21 days after the first cycle. (Browder et al. Can Res 60:1878-1886, 2000). Similarly, the anti-HER-2 monoclonal antibody, trastuzumab, is administered to HER-2 positive breast cancer patients in one larger initial dose (4 mg/kg) given over period of about 90 minutes, followed by smaller weekly maintenance doses (2 mg/kg) that are administered over a shorter period of time, about 30 minutes. When administered in conjunction with other adjuvant cancer therapies (e.g., chemotherapy, hormone therapy), the anti-HER-2 monoclonal antibody is administered on the same or similar cycles as the other cancer therapy.

An anti-tumor effective amount of β₅ integrin antagonist which directly inhibits the expression or activity of a β₅ integrin subunit in a tumor cell (e.g., neutralizing antibodies (e.g., KN52, P5H9, B5-IVF2), inhibitory nucleic acids (e.g., siRNA, antisense nucleotides)) can be administered, for example, in a first cycle in which the maximum tolerated dose of the antagonist is administered in one interval/dose, or in several closely spaced intervals (minutes, hours, days) with another/second cycle administered after a suitable off-therapy period (e.g., one or more weeks). Suitable dosing schedules and amounts for a β₅ integrin antagonist can be readily determined by a clinician of ordinary skill. Decreased toxicity of a particular β5 integrin antagonist as compared to chemotherapeutic agents can allow for the time between administration cycles to be shorter. When used as an adjuvant therapy (to, e.g., surgery, radiation therapy, other primary therapies), an anti-tumor effective amount of a β₅ integrin antagonist is preferably administered on a dosing schedule that is similar to that of the other cancer therapy (e.g., chemotherapeutics), or on a dosing schedule determined by the skilled clinician to be more/most effective at inhibiting (reducing, preventing) tumor growth. A treatment regimen for an anti-tumor effective amount of a β₅ integrin antagonist (e.g., an antibody) can be about 1 mg/kg to about 10 mg/kg (preferably about 4 mg/Kg to about 10 mg/Kg) administered initially in a single dose followed by about 1 mg/kg to about 10 mg/kg (preferably about 4 mg/Kg to about 10 mg/Kg) administered after a period of about 1, 2, 3 or 4 weeks (e.g., administered every 2 to about 4 weeks over a period of about 4 to about 6 months).

Accordingly, one aspect of the invention also relates to a method for directly inhibiting the growth of a tumor that expresses a β₅ integrin comprising administering to a patient with the tumor a therapeutically effective amount (e.g., an anti-tumor effective amount) of a β₅ integrin antagonist. Preferably, the β₅ integrin antagonist does not bind an αᵥβ₃ heterodimer. In one embodiment, the β₅ integrin antagonist directly inhibits the growth of the tumor by inducing the apoptosis of the tumor cells or by inhibiting the proliferation of the tumor cells. The β₅ integrin antagonist can inhibit the expression (e.g., siRNA, antisense oligonucleotides) or activity (e.g., antibody, peptide (e.g., RGD peptide), peptide mimetic) of a β₅ integrin or β₅ integrin heterodimer, thereby directly inhibiting the growth of the cells of the tumor.

In another embodiment, an “anti-angiogenic effective amount” of a β₅ integrin antagonist is administered to a patient in need thereof. Anti-angiogenic therapies may indirectly affect (inhibit, reduce) tumor growth by blocking the formation of new blood vessels that supply tumors with nutrients needed to sustain tumor growth and enable tumors to metastasize. Starving the tumor of nutrients and blood supply in this manner can eventually cause the cells of the tumor to die by necrosis and/or apoptosis. Previous work has indicated that the clinical outcomes (inhibition of endothelial cell-mediated tumor angiogenesis and tumor growth) of cancer therapies that involve the blocking of angiogenic factors (e.g., VEGF, bFGF, TGF-α, IL-8, PDGF) or their signaling have been more efficacious when lower dosage levels are administered more frequently, providing a continuous blood level of the antiangiogenic agent. (See Browder et al. Can. Res. 60:1878-1886, 2000; Folkman J., Sem. Can. Biol. 13:159-167, 2003). This type of dosing can be referred to as an “anti-angiogenic” or “metronomic” schedule. This anti-angiogenic dosing schedule is in contrast to the high dose, cyclic treatment regimen used for therapies that directly inhibit tumor growth. An anti-angiogenic treatment regimen has been used with a targeted inhibitor of angiogenesis (thrombospondinl and platelet growth factor-4 (TNP-470)) and the chemotherapeutic agent cyclophophamide. Every 6 days, TNP-470 was administered at a dose lower than the maximum tolerated dose and cyclophophamide was administered at a dose of 170 mg/kg. Id. This treatment regimen resulted in complete regression of the tumors. Id. In fact, anti-angiogenic treatments are most effective when administered in concert with other anti-cancer therapeutic agents; for example, those agents that directly inhibit tumor growth (e.g., chemotherapeutic agents). Id.

Accordingly, an anti-angiogenic effective amount of a β₅ integrin antagonist that is, for example, an antibody, can be from about 0.1 mg/kg to about 3 mg/kg every 1 to 7 days over a period of about 4 to about 6 months.

The antiangiogenic effective amount of a β₅ integrin antagonist can be administered alone, as an adjuvant therapy to a primary cancer therapy (surgery, radiation), with anti-angiogenic therapies (e.g., avastatin, endostatin, tumstatin, angiostatin) or as a primary therapy with other adjuvant therapies (e.g., chemotherapeutic, hormone).

### Other Therapies

Many anti-cancer therapeurics (e.g., targeted cancer therapeutics) are administered in conjunction with one or more other therapeutics and/or treatments regimens. For example, a therapeutic agent used as an adjuvant therapy can be administered as a secondary therapy to some primary cancer therapy. Adjuvant therapies include, for example, chemotherapies (e.g., dacarbazine (DTIC), Cis-platinum, cimetidine, tamoxifen, cyclophophamide), hormone (endocrine) therapies (e.g., anti-estrogen therapy, androgen deprivation therapy (ADT), luteinizing hormone-releasing hormone (LH-RH) agonists, aromatase inhibitors (Als, such as anastrozole, exemestane, letrozole), estrogen receptor modulators (e.g., tamoxifen, raloxifene, toremifene)) and radiation therapy. Radiation therapy can be used as both a primary and adjuvant therapy. Although occasionally used alone, these therapies are typically used as adjuvants, that is, in addition to primary cancer treatments like the surgical removal of tumors, radiation therapy or antibody therapy (e.g., a monoclonal antibody administered alone and/or conjugated to a cytotoxic agent (e.g., ricin)). Numerous other therapies can also be administered during a cancer treatment regime to mitigate the effects of the disease and/or side effects of the cancer treatment including therapies to manage pain (narcotics, acupuncture), gastric discomfort (antacids), dizziness (anti-veritgo medications), nausea (anti-nausea medications), infection (e.g., medications to increase red/white blood cell counts) and the like.

Thus, a β₅ integrin antagonist can be administered as an adjuvant therapy (e.g., with another primary cancer therapy or treatment). As an adjuvant therapy, the β₅ integrin subunit antagonist can be administered before, after or concurrently with a primary therapy like radiation and/or the surgical removal of a tumor(s). In some embodiments, the method further comprises administering a therapeutically effective amount of a β₅ integrin antagonist and one or more other therapies (e.g., adjuvant therapies, other targeted therapies). An adjuvant therapy (e.g., a chemotherapeutic agent) and/or the one or more other targeted therapies and the β₅ integrin antagonist can be co-administered simultaneously (i.e., concurrently) as either separate formulations or as a joint formulation. Alternatively, the therapies can be administered sequentially, as separate compositions, within an appropriate time frame (e.g., a cancer treatment session/interval (e.g., 1.5 to 5 hours)) as determined by the skilled clinician (e.g., a time sufficient to allow an overlap of the pharmaceutical effects of the therapies). The adjuvant therapy and/or one or more other targeted therapies and the β₅ integrin subunit antagonist can be administered in a single dose or multiple doses in an order and on a schedule suitable to achieve a desired therapeutic effect (e.g., inhibition of tumor growth).

### HER-2 Positive Tumor Therapy

Tumors over-expressing the HER-2 protein, generally due to the presence of extra copies of the HER-2 gene in the tumor cells, are defined as HER-2 positive tumors. Overexpression of the HER-2 protein, which can be determined immunohistochemically in cultured, biopsied or surgical tumor tissue samples (or using other suitable methods), is associated with more aggressive tumor growth and progression. In HER-2 positive breast cancers, a therapy which inhibited HER-2 activity (e.g., an anti-HER-2 antibody), when used in conjunction with other adjuvant therapies (e.g., chemotherapy, hormone therapy) reduced the risk of cancer recurrence or death by about half (Romond E. H. et al. N. Engl. J Med. 353(16):1673-1684, 2005). Therapies which target HER-2 expression or activity include, for example, monoclonal antibodies (e.g., trastuzumab (Herceptin®, Gcnetech, Inc.)), small molecule compounds and antisense HER-2 oligonucleotides. An indication for anti-HER-2 therapy can be confirmed using any suitable methods such as fluorescence in situ hybridization (FISH) which can be used to detect the presence of excess copies of the HER-2 gene in the tumor cells, or immunohistochemistry.

Accordingly, in one embodiment of the method for treating cancer in a mammalian subject by administering to the subject a therapeutically effective amount of a β₅ integrin subunit antagonist further comprises administering a HER-2 antagonist, such as trastuzumab.

Another aspect of the invention provides for a method of treating a HER-2 positive tumor comprising administering to a mammalian subject a therapeutically effective amount of a β₅ integrin subunit antagonist and a therapeutically effective amount of a HER-2 antagonist, such as trastuzumab. In some embodiments, the HER-2 positive tumor is also β₅ integrin positive. In a particular embodiment, the tumor is a HER-2 positive breast cancer tumor that also expresses a β₅ integrin. Several types of breast cancer tumors (e.g., carcinoma) can be treated with the anti-HER-2 and anti-β₅ integrin subunit therapy, including ductal breast cancer, lobular breast cancer and nipple breast cancer.

When a β₅ integrin subunit antagonist and a HER-2 antagonist (e.g., trastuzumab) are coadministered, for example, to treat cancer, a HER-2⁺ tumor, a HER-2⁺ β35 integrin⁺ tumor, a HER-2⁺ β₅ integrin⁺ ER⁻ tumor, it is preferred that a synergistically effective amount of the two agents are administered. A clinician of ordinary skill can readily determine appropriate amounts of each agent to achieve synergy (CI<1).

In yet another aspect, the invention provides for a composition comprising a HER-2 antagonist and a β₅ integrin subunit antagonist, e.g., a pharmaceutical composition and/or formulation comprising two or more therapeutic agents (e.g., a β₅ integrin antagonist and HER-2 antagonist) and a pysiologically or pharmaceutically acceptable carrier. In one embodiment, the composition comprises a β₅ integrin antagonist, a HER-2 antagonist and a pysiologically or pharmaceutically acceptable carrier. In another embodiment, the composition comprises trastuzumab, a β₅ integrin antagonist and a pysiologically or pharmaceutically acceptable carrier. In another embodiment, the composition comprises an antibody which selectively binds a β₅ integrin (e.g., KN52, B5-IVF2 antibodies) or a β5 heterodimer (e.g., a αᵥβ₅ heterodimer, (e.g., P5H9 antibody)), a HER-2 antagonist and a pysiologically or pharmaceutically acceptable carrier. In yet another embodiment, the composition comprises trastuzumab, an antibody which selectively binds a β₅ integrin and a pysiologically or pharmaceutically acceptable carrier. Pharmaceutical compositions and formulations are discussed herein.

When the composition of the invention comprises a β₅ integrin subunit antagonist and a HER-2 antagonist (e.g., trastuzumab) it is preferred that the composition contains a synergistically effective amount of the two agents.

### Luminal A Subtype Breast Cancer Therapy

In another aspect, the invention provides for a method of treating a luminal A subtype breast cancer tumor comprising administering to a mammalian subject a therapeutically effective amount of a β₅ integrin antagonist. The method can further comprise administering a therapy used for the treatment of luminal A subtype of breast cancer. Luminal A subtype breast cancers are estrogen receptor and progesterone receptor positive. Thus, therapies to treat luminal A subtypes of breast cancer can include those that target these hormonal receptors (e.g., hormonal/endocrine therapies), as described herein.

### Modes of Administration

According to the methods of the invention, a therapeutically effective amount (anti-tumor effective amount, anti-angiogenesis effective amount) is administered to a mammalian subject to treat cancer in a mammalian subject. The term “mammalian subject” is defined herein to include mammals such as primates (e.g., humans) cows, sheep, goats, horses, dogs cats, rabbits, guinea pigs, rats, mice or other bovine, ovine, equine, canine feline, rodent or murine species.

Agents that act as β₅ integrin antagonists can be administered in single or multiple doses. Suitable dosing and regimens of administration can be determined by a practitioner and are dependent on the agent(s) chosen, pharmaceutical formulation and route of administration, various patient factors and other considerations. With respect to the administration of a β₅ integrin antagonist with one or more other therapies or treatments (adjuvant, targeted, cancer treatment-associated) the β₅ integrin antagonist is typically administered as a single dose (by e.g., injection, infusion), followed by repeated doses at particular intervals (e.g., one or more hours).

The amount of the β₅ integrin antagonist to be administered (e.g., therapeutically effective amount, anti-tumor effective amount, anti-angiogenesis effective amount) can be determined by a clinician using the guidance provided herein and other methods known in the art and is dependent on several factors including, for example, the particular agent chosen, the subject's age, sensitivity, tolerance to drugs and overall well-being. For example, suitable dosages for antibodies can be from about 0.1 mg/kg to about 300 mg/kg body weight per treatment and preferably from about 0.01 mg/kg to about 100 mg/kg body weight per treatment. Preferably, the dosage does not cause or produces minimal adverse side effects (e.g., immunogenic response, nausea, dizziness, gastric upset, hyperviscosity syndromes, congestive heart failure, stroke, pulmonary edema). Where the β₅ integrin antagonist is a polypeptide (linear, cyclic, mimetic) or small molecule, the preferred dosage will result in a plasma concentration of the peptide from about 0.1 μg/mL to about 200 μg/mL. Determining the dosage for a particular agent, patient and cancer is well within the abilities of one of skill in the art.

A variety of routes of administration can be used including, for example, oral, dietary, topical, transdermal, rectal, parenteral (e.g., intravenous, intraaterial, intramuscular, subcutaneous injection, intradermal injection), intravenous infusion and inhalation (e.g., intrabronchial, intranasal or oral inhalation, intranasal drops) routes of administration, depending on the agent and the particular cancer to be treated. Administration can be local or systemic as indicated. The preferred mode of administration can vary depending on the particular agent chosen; however, intravenous infusion is generally preferred (e.g., to administer neutralizing β₅ integrin antibodies).

The agent (β5 integrin antagonist) can be administered to a mammalian subject as part of a pharmaceutical or physiological composition. For example, the agent can be administered as part of a pharmaceutical composition for inhibition of β5 integrin activity and a pharmaceutically acceptable carrier. Formulations or compositions comprising a β5 integrin antagonist or compositions comprising a β5 integrin antagonist and one or more other targeted therapies (e.g., a HER-2 antagonist) will vary according to the route of administration selected (e.g., solution, emulsion or capsule). Suitable pharmaceutical carriers can contain inert ingredients which do not interact with the β5 integrin antagonist. Standard pharmaceutical formulation techniques can be employed, such as those described in Remington's Pharmaceutical Sciences, Mack Publishing Company, Easton, Pa. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's lactate and the like. Formulations can also include small amounts of substances that enhance the effectiveness of the active ingredient (e.g., emulsifying, solubilizing, pH buffering, wetting agents). Methods of encapsulation compositions (such as in a coating of hard gelatin or cyclodextran) are known in the art. For inhalation, the agent can be solubilized and loaded into a suitable dispenser for administration (e.g., an atomizer or nebulizer or pressurized aerosol dispenser).

For example, nucleic acid-based β₅ integrin antagonists (e.g., siRNAs, antisense oligonucleotides, natural or synthetic nucleic acids, nucleic acid analogs) can be introduced into a mammalian subject of interest in a number of ways. For instance, chemically synthesized or in vitro transcribed nucleic acids can be transfected into cells in cell culture by any suitable method (e.g., viral infection). The nucleic acids may also be expressed endogenously from expression vectors or PCR products in host cells or packaged into synthetic or engineered compositions (e.g., liposomes, polymers, nanoparticles) that can then be introduced directly into the bloodstream of a mammalian subject (by, e.g., injection, infusion). Anti-β₅ integrin nucleic acids or nucleic acid expression vectors (e.g., retroviral, adenoviral, adeno-associated and herpes simplex viral vectors, engineered vectors, non-viral-mediated vectors) can also be introduced into a mammalian subject directly using established gene therapy strategies and protocols (see e.g., Tochilin V. P. Annu Rev Biomed Eng 8:343-375, 2006; Recombinant DNA and Gene Transfer, Office of Biotechnology Activities, National Institutes of Health Guidelines).

Similarly, where the agent is a protein or polypeptide, the agent can be administered via in vivo expression of recombinant protein. In vivo expression can be accomplished by somatic cell expression according to suitable methods (see, e.g., U.S. Pat. No. 5,399,346). Further, a nucleic acid encoding the polypeptide can also be incorporated into retroviral, adenoviral or other suitable vectors (preferably, a replication deficient infectious vector) for delivery, or can be introduced into a transfected or transformed host cell capable of expressing the polypeptide for delivery. In the latter embodiment, the cells can be implanted (alone or in a barrier device), injected or otherwise introduced in an amount effective to express the polypeptide in a therapeutically effective amount.

### Diagnostic Methods

The present invention also relates to method for identifying a candidate for an anti-cancer therapy using a β₅ integrin antagonist comprising providing a tumor sample obtained from a subject and assessing expression of β₅ integrin in the tumor sample, wherein expression of β₅ integrin by the tumor or increased expression of β₅ integrin by the tumor relative to a suitable control, indicates that the subject is a candidate for an anti-cancer therapy using a β₅ integrin antagonist.

The tumor sample can be an suitable available sample or, optionally, one that is obtained from an individual. Suitable tumor samples, include a tissue sample, a biological fluid sample, a cell(s) (e.g., tumor) sample, and the like. Any means of sampling from a subject, for example, by blood draw, spinal tap, tissue smear or scrape, or tissue biopsy can be used to obtain a sample. Thus, the sample can be a biopsy specimen (e.g, tumor, polyp, mass (solid, cell)), aspirate, smear or blood sample. The sample can be from a tissue that has a tumor (e.g., cancerous growth) and/or tumor cells, or is suspecting of having a tumor and/or tumor cells. For example, a tumor biopsy can be obtained in an open biopsy, a procedure in which an entire (excisional biopsy) or partial (incisional biopsy) mass is removed from a target area. Alternatively, a tumor sample can be obtained through a percutaneous biopsy, a procedure performed with a needle-like instrument through a small incision or puncture (with or without the aid of a imaging device) to obtain individual cells or clusters of cells (e.g., a fine needle aspiration (FNA)) or a core or fragment of tissues (core biopsy). The biopsy samples can be examined cytologically (e.g., smear), histologically (e.g., frozen or paraffin section) or using any other suitable method (e.g., molecular diagnostic methods). A tumor sample can also be obtained by in vitro harvest of cultured human cells derived from an individual's tissue. Tumor samples can, if desired, be stored before analysis by suitable storage means that preserve a sample's protein and/or nucleic acid in an analyzable condition, such as quick freezing, or a controlled freezing regime. If desired, freezing can be performed in the presence of a cryoprotectant, for example, dimethyl sulfoxide (DMSO), glycerol, or propanediol-sucrose. Tumor samples can be pooled, as appropriate, before or after storage for purposes of analysis.

Suitable assays can be used to assess the presence or amount of a β₅ integrin in a sample (e.g., biological sample). Methods to detect a β₅ integrin protein or peptide can include immunological and immunochemical methods like flow cytometry (e.g., FACS analysis), enzyme-linked immunosorbent assays (ELISA), including chemiluminescence assays, radioimmunoassay, immunoblot (e.g., Western blot), and immunohistology, or other suitable methods such as mass spectroscopy. For example, antibodies to β₅ integrin can be used to determine the presence and/or expression level of β₅ integrin in a sample directly or indirectly using, for instance, immunohistology. For instance, paraffin sections can be taken from a biopsy, fixed to a slide and combined with one or more antibodies by suitable methods.

Methods to detect a β₅ integrin gene or expression thereof (e.g., DNA, mRNA) include β₅ integrin nucleic acid amplification and/or visualization. To detect a β₅ integrin gene or expression thereof, nucleic acid can be isolated from an individual by suitable methods which are routine in the art (see, e.g., Sambrook et al., 1989). Isolated nucleic acid can then be amplified (by e.g., polymerase chain reaction (PCR) (e.g., direct PCR, quantitative real time PCR, reverse transcriptase PCR), ligase chain reaction, self sustained sequence replication, transcriptional amplification system, Q-Beta Replicase, or the like) and visualized (by e.g., labeling of the nucleic acid during amplification, exposure to intercalating compounds/dyes, probes). β₅ integrin gene or expression thereof can also be detected using a nucleic acid probe, for example, a labeled nucleic acid probe (e.g., fluorescence in situ hybridization (FISH)) directly in a paraffin section of a tissue sample taken from, e.g., a tumor biopsy, or using other suitable methods. β₅ integrin gene or expression thereof can also be assessed by Southern blot or in solution (e.g., dyes, probes). Further, a gene chip, microarray, probe (e.g., quantum dots) or other such device (e.g., sensor, nanonsensor/detector) can be used to detect expression and/or differential expression of a β₅ integrin gene.

The presence or absence of β₅ integrin can be ascertained by the methods described herein or other suitable assays. An increase in expression of β₅ integrin can be determined by comparison of β₅ integrin expression in the sample to that of a suitable control. Suitable controls include, for instance, a non-neoplastic tissue sample from the individual, non-cancerous cells, non-metastatic cancer cells, non-malignant (benign) cells or the like, or a suitable known or determined standard. The control can be a known or determined typical, normal or normalized range or level of expression of a β₅ integrin protein or gene (e.g., an expression standard). thus, the method does not require that expression of the gene/protein be assessed in a suitable control. β₅ integrin expression can be compared to its expression in known or determined standard.

The present invention will now be illustrated by the following Examples, which are not intended to be limiting in any way.

### Exemplification

### Material and Methods

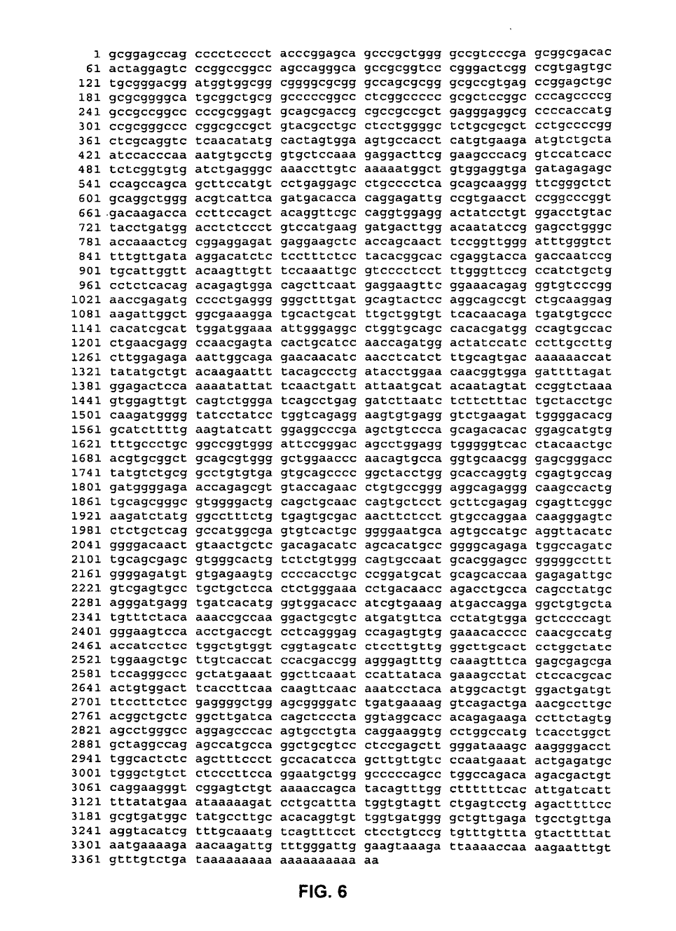

siRNAs and reagents: The following siRNAs (Dharmacon, CO, USA) targeting the human β5 integrin gene (ITGB5) (SEQ ID NO:1) (Genbank Accession No. NM 002213, see also FIG. 6) were used:

| | | |
|---|---|---|
| siRNA1: | 5′-GAACAACGGUGGAGAUUUU-3′; | (SEQ ID NO: 3) |
| siRNA2: | 5′-GGAGGGAGUUUGCAAAGUU-3′; | (SEQ ID NO: 4) |
| siRNA3: | 5′-GCUCGCAGGUCUCAACAUA-3′; | (SEQ ID NO: 5) |
| siRNA4: | 5′-GGGAUGAGGUGAUCACAUG-3′. | (SEQ ID NO: 6) |

Control siRNAs, including siCONTROL non-targeting siRNA (siCTRL), siCONTROL non-targeting siRNA pool (siPOOL) and siCONTROL TOX™ transfection control (siTOX) were also purchased from Dharmacon. Transfection reagent Lipofectamine 2000 was purchased from lnvitrogen Canada, Burlington, ON, Canada. The reagents for the Sulforhodamine B (SRB) assay were from Sigma Canada, Oakville, ON, Canada. The β5 integrin subunit antibodies KN52 (eBiosciences, Inc.), P5H9 (R & D Systems) and B5-IVF2 (Abcam, PLC) were purchased from commercial providers as was the isotype IgG1 control antibody (eBiosciences, Inc.).

Transfection of siRNAs into breast/colon cancer cell lines: Different breast and colon cancer cell lines were used for siRNA transfection. Cells were seeded at various concentrations, ranging from 1500 to 6000 per well according to cell growth rate, into 96 well plates. 40 nM individual siRNAs or 40 nM siRNA pool including four individual siRNAs at 10 nM each were transfected into cells using Lipofectamine2000 24 hrs after cell seeding. Cells were then incubated at 37° C. for five days before cell viability assay were conducted.

Sulforhodamine B (SRB) assay: SRB assay was performed to assess cell survival. SRB is a water-soluble dye that binds to the basic amino acids of the cellular proteins. Thus, colorimetric measurement of the bound dye provides an estimate of the total protein mass, is related to the cell number. The cells were fixed in situ by gently aspirating off the culture media and adding 50 μl ice cold 10% Tri-chloroacetic Acid (TCA) per well and incubate at 4° C. for 30-60 min. The plates were washed with tap water five times and allowed to air dry for 5 min. 50 μl 0.4% (w/v) Sulforhodamine B solution in 1% (v/v) acetic acid was added per well and incubated for 30 min at RT for staining. Following staining, plates were washed four times with 1% acetic acid to remove any unbound dye and then allowed to air dry for 5 min. The stain was solubilized with 100 μl of 10 mM Tris pH 10.5 per well. Absorbance was read at 570 nm. The cell survival percentage after each siRNA(s) knock down was calculated over the non-silencing control siCTRL or siPOOL as well as siTOX as a transfection efficiency control.

Cell growth inhibition. MCF-7 cells purchased from ATCC were suspended in D-MEM containing 2% FCS and then seeded in a 96-well plate at 4×10⁴/mL and 0.1 mL/well. Mouse monoclonal anti-integrin beta 5 antibody from eBioscience was added into the well immediately to reach a final concentration of 10 ug/mL. A non-specific IgG1 of the same isotype was used at the same concentration to serve as a negative control. The experiment was done in triplicates. The number of MCF-7 cells in each well was measured using alamarBlue™ (Biosource) on day 1, day 2, day 3 and day 4 post-treatment. The statistical significance was determined using Student's t test.

Multiple cancer cell lines (lung cancer cell lines: A549, H358; adherent breast cancer cell lines: MDA-MB-468, MDA-MB-435, MDA-MB-231, MCF-7, T47D, BT-474, and one suspension breast cancer cell line: Colo824) were suspended in D-MEM containing 2% FCS and then seeded onto a 96-well plate at 4×10⁴/mL and 0.1 mL/well. Mouse monoclonal anti-integrin beta 5 antibody from eBioscience was added into the well immediately to reach a final concentration of 10 ug/mL. A non-specific IgG1 of the same isotype was used at the same concentration to serve as a negative control. The experiment was done in triplicate. The number of cells in each well was measured using alamarBlue™, combined with salforhodamine B (SRB) or an automatic cell counter (Beckman Coulter) on day 4 post-treatment. The statistical significance was determined using Student's t test.

### EXAMPLE 1

### β₅ Integrin Expression in Cancer Cells

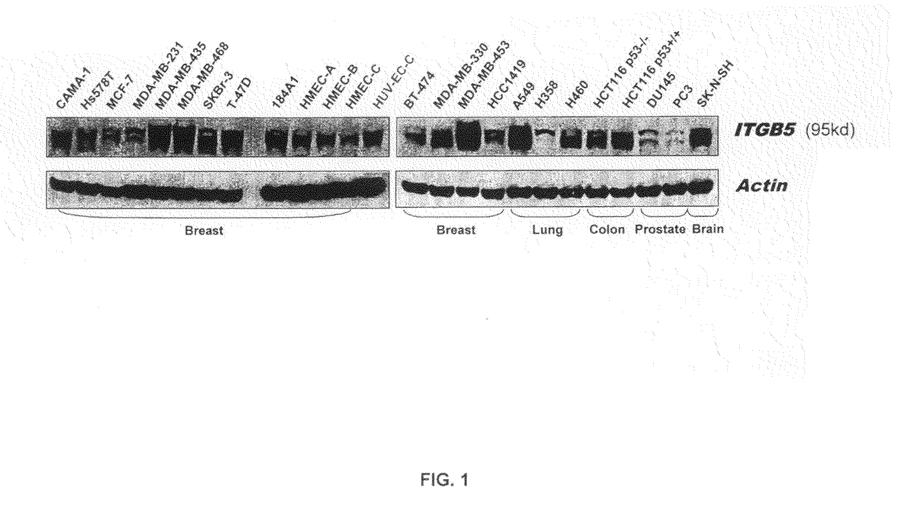

Western blot analysis with an anti-β5 integrin subunit monoclonal antibody revealed that the β5 integrin subunit protein was expressed in multiple cancer cells originating from different tissue types, including breast, lung, colon and brain (FIG. 1). In particular, higher levels of the protein were detected in breast cancer cells Cama-1, MDA-MB-435, MDA-MB-468, T-47D, MDA-MB-330, MDA-MB-453 compared to normal human mammary epithelial cells (HMEC) and high levels of the protein were also detected in A549 (lung tumor), HCT116 (colon tumor) and SK-N-SH (brain tumor) cells. These results indicate that β₅ integrin is a target for cancer therapy.

### EXAMPLE 2

### Validation of β₅ Integrin as an Anti-Tumor Target

To validate β₅ integrin s an anti-tumor target, the biological effects of β₅ integrin antagonists were investigated.

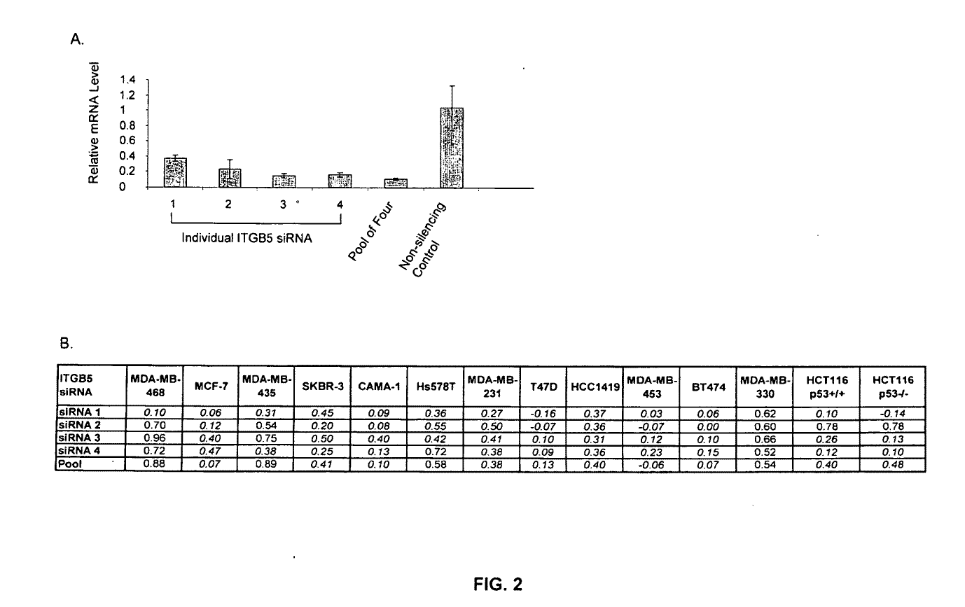

MDA MB-468 cells were transfected with 40 nM of individual ITGB5 siRNA or a pool of the 4 siRNAs. The total RNAs were then isolated 72 hours post transfection and the ITGB5 mRNA levels were determined by qRTPCT. The data were normalized over the beta actin mRNA. All siRNAs tested showed better than 60% knockdown (FIG. 2A).

Various breast and colon cancer cells were transfected with either 4 distinct siRNAs individually that target the β₅ integrin or the pool of the 4 siRNAs. At day 5 post-transfection, the growth of the cells were determined using SRB assay that measures protein content of the cells. The data were normalized over a non-silencing control as well as a toxic siRNA control for transfection efficiency and represent the percentage of cell remaining. Molecular depletion of the β₅ integrin gene expression in cancer cells substantially inhibited the growth of the cancer cells (see FIG. 2B).

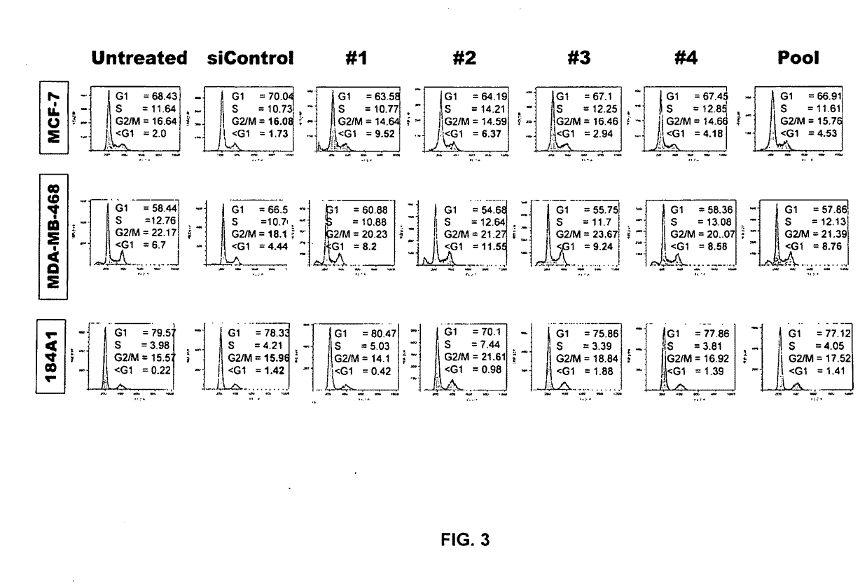

Cancer cells (MCF7 and MDA-MB-468) or normal cells (184A1) were transfected with 40 nM of ITGB5 siRNAs individually or as a pool of 4, or a non-silencing siRNA control. At day 3 post transfection, the cells were analyzed by flow cytometry to reveal the cell death caused by β₅ integrin siRNA. The data showed that depletion of the β₅ integrin subunit gene led to significant cell death in cancer cells (sub G1 population) (see FIG. 3). Little effect was seen when the β5 integrin subunit gene was depleted in the normal cells (184A1).

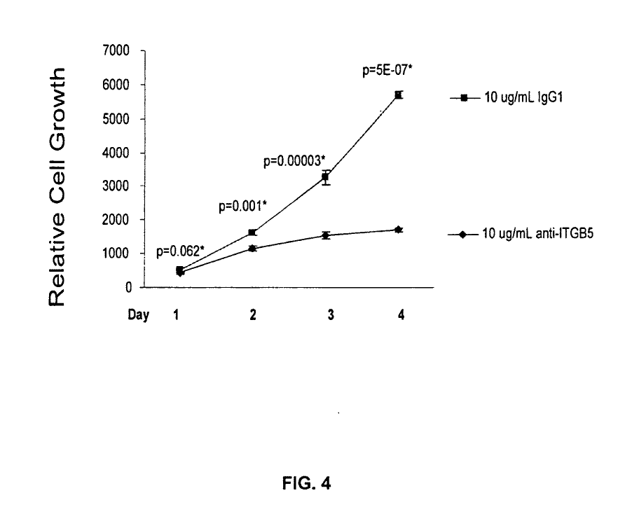

MCF7 breast cancer cells were treated with either 10 ug/ml of an anti β₅ integrin subunit monoclonal antibody (KN52, eBiosciences) or an IgG1 isotype control antibody (eBiosciences). Cell growth was then determined by alamarBlue™ assay at day 1, 2, 3 and 4 post treatment. The anti-β5 integrin subunit antibody significantly inhibited the growth of MCF7 cells (FIG. 4).

Multiple cancer cell lines, including cancer cells of breast, prostate, brain, ovary lung and colon were treated with 10 μg/ml of an anti-β₅ integrin antibody (KN52, eBiosciences) or an IgG1 isotype-matched control (eBiosciences) for 4 days. Cell proliferation was then measured using an alamarBlue™ assay and SRB or cell counting (Table 1). The data represent the percentage of cells inhibited after 4 day-treatment with the antibody compared to the isotype control. The β₅ integrin antibody treatment led to inhibition of cancer cell growth. In contrast, the same antibody had little effect on a normal cell line (184A1) or normal human mammary epithelial cells (HMEC C).

TABLE 1
β₅ integrin inhibits the proliferation of cancer cells.
BREAST

| | | | | | | | | | | | | | |
|---|---|---|---|---|---|---|---|---|---|---|---|---|---|
| Cell | MCF7 | 468 | HCC- | 435 | T47D | SKBr3 | 453 | 231 | HCC- | Du- | BT- | 184- | HMEC |
| line | | | 1954 | | | | | | 1419 | 4475 | 474 | A1 | C |
| Inhib- | 49.07% | 44.31% | 39.34% | 33.69% | 30.86% | 26.18% | 24.88% | 23.03% | 20.88% | 2.13% | 0% | 0% | 0% |
| ition | | | | | | | | | | | | | |

| | | | |
|---|---|---|---|
| PROSTATE | BRAIN | OVARY | LUNG |

| | | | | | | | | | | |
|---|---|---|---|---|---|---|---|---|---|---|
| Cell | PC3 | Du145 | A172 | SK-N-SH | CaoV3 | SW626 | Skov3 | A549 | H460 | H358 |
| line | | | | | | | | | | |
| Inhib- | 11.16% | 17.07% | 90% | 40% | 45.89% | 29.70% | 15.13% | 60%-80% | 39.72% | 30.14% |
| ition | | | | | | | | | | |

COLON

| | | | | | | |
|---|---|---|---|---|---|---|
| Cell | HCT15 | SW620 | HCT8 | SW480 | Colo205 | SW1783 |
| line | | | | | | |
| Inhib- | 58.69% | 48.54% | 33.01% | 30.22% | 27.24% | 26.56% |
| ition | | | | | | |

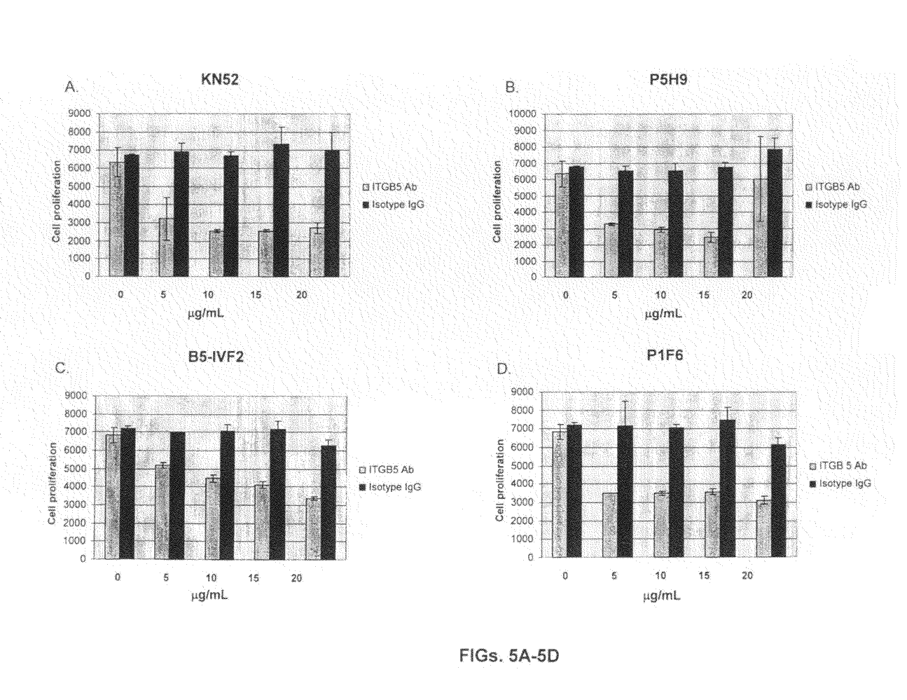

Growth inhibition assays were performed as previously described (SRB assay) with additional anti-β₅ integrin antibodies (KN52 (eBioSciences), P5H9 and B5-IVF2). Cell proliferation was measured using the alamarBlue™ assay. The additional antibodies to the β₅ integrin also inhibited cancer cell growth (FIGS. 5A-5D).

### EXAMPLE 3

### β₅ Integrin and HER-2 Overexpression on Breast Cancer Cells

The inventors have discovered an association of β₅ overexpression with HER-2 overexpression on breast tumors. The expression level of the of β₅ integrin transcript in breast tumor tissues were compared to its expression in normal breast tissue. β₅ integrin subunit expression was up-regulated in 25% of the HER-2 positive/estrogen receptor (ER) negative subtype of breast cancers (see Table 2) (Perou et al., Nature 406(6797):747-752, 2000). β₅ integrin expression was also found to be up-regulated in about 18% of luminal A subtype breast cancers as compared to its expression in normal breast tissue (Table 2).

TABLE 2
β5 integrin subunit expression in breast cancer tumors expressing known
biomarkers.

| % | | | % | % |
|---|---|---|---|---|
| increase | % | % increase | increase | increase |
| All | increase | HER2+/ | Luminal | Luminal |
| cancers | Basal-like | ER− | A | B |
| 10 | 0 | 25 | 18 | 6 |

### EXAMPLE 4

### Synergy in Targeting β5 Integrin and HER-2

The SKBr3 breast cancer cell line (HER-2⁺) was treated with a combination of HERCEPTIN® (trastuzumab, Genentech) and anti-β5 integrin subunit antibody in culture for 5 days under normoxia conditions, and cell growth was then measured to determine % inhibition of cell growth.

SKBr-3 cells were seeded at 5000/well in 96 well plates with culture medium (McCoy's 5A+2 mM L-glutame+3% FBS) and incubated at 37° C., 5% CO₂ overnight. Trastuzumab (Genentech) or/and anti-ITGB5 antibody (KN52, eBiosciences) at concentrations of 0, 0.08, 0.4, 2, 10, 50 μg/ml were added and the cells were incubated further at 37° C., 5% CO₂ for 5 days. Cell proliferation was measured using the SRB assay.

The results are shown in Table 3. The data was analyzed and the combination index (CI) values were calculated using CalcuSyn software (Biosoft; See, Chou, T-C., Theoretical Basis, Experimental Design, and Computerized Simulation of Synergism and Antagonism in Drug Combination Studies, Pharmacological Reviews 58(3):621-68l (2006)). CI values of <1 indicate synergy. CI values of <0.1 indicate very strong synergism. (Id.) The CI values for various amounts of trastuzumab and anti-β5 integrin subunit antibody are shown in Table 4, and show that there was very strong synergy at each combination tested.

TABLE 3
% Inhibtion of cell growth

| anti β₅ integrin chain | trastuzumab (μg/ml) | | | | | |
|---|---|---|---|---|---|---|
| antibody (μg/ml) | 0 | 0.08 | 0.4 | 2 | 10 | 50 |
| 0 | 0.00 | 31.23 | 50.67 | 54.44 | 53.87 | 55.59 |
| 0.08 | 30.01 | 59.29 | 66.97 | 67.76 | 66.82 | 67.80 |
| 0.4 | 43.72 | 64.90 | 71.09 | 76.35 | 77.58 | 78.70 |
| 2 | 52.65 | 76.72 | 80.00 | 82.43 | 81.94 | 84.12 |
| 10 | 71.52 | 85.15 | 88.26 | 87.51 | 87.90 | 87.14 |
| 50 | 76.23 | 86.01 | 89.80 | 89.58 | 88.88 | 91.73 |

TABLE 4

| trastuzumab | anti β5 integrin chain antibody | |
|---|---|---|
| (μg/ml) | (μg/ml) | CI |
| 0.08 | 0.08 | 0.028 |
| 0.4 | 0.4 | 0.025 |
| 2 | 2 | 0.018 |
| 10 | 10 | 0.021 |
| 50 | 50 | 0.026 |
| 0.08 | 0.4 | 0.059 |
| 0.4 | 2 | 0.027 |
| 2 | 10 | 0.021 |
| 10 | 50 | 0.077 |
| 0.4 | 0.08 | 0.010 |
| 2 | 0.4 | 0.011 |
| 10 | 2 | 0.018 |
| 50 | 10 | 0.028 |

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1. A method for treating β₅ integrin positive cancer in a mammalian subject comprising administering to the subject a therapeutically effective amount of a β₅ integrin antagonist.

2. The method of claim 1, wherein the cancer is selected from the group consisting of breast cancer, lung cancer, colon cancer, prostate cancer and brain cancer.

3-4. (canceled)

5. The method of claim 4, wherein said β₅ integrin antagonist is selected from the group consisting of an antibody or antigen-binding fragment of an antibody, a small interfering ribonucleic acid (siRNA), a peptide, a peptidemimetic, an antisense oligonucleotide, and a small molecule.

6. The method of claim 1, further comprising administering one or more other treatments.

7. The method of claim 6, wherein said cancer is HER-2 positive and a HER-2 antagonist is also administered.

8. The method of claim 7, wherein said HER-2 antagonist is trastuzumab.

9. A method for directly inhibiting the growth of a tumor that expresses a β₅ integrin comprising administering to a patient with said tumor a therapeutically effective amount of a β₅ integrin antagonist.

10-11. (canceled)

12. The method of claim 11, wherein said β₅ integrin antagonist is selected from the group consisting of an antibody or antigen-binding fragment of an antibody, a small interfering ribonucleic acid (siRNA), a peptide, a peptidemimetic, an antisense oligonucleotide, and a small molecule.

13. A method of treating a β₅ integrin positive metastatic tumor by administering to a mammalian subject a therapeutically effective amount of a β₅ integrin antagonist.

14-24. (canceled)

25. A method of treating a luminal A subtype breast cancer tumor comprising administering to a mammalian subject a therapeutically effective amount of a β₅ integrin antagonist.

26. The method of claim 25, wherein said luminal A subtype tumor also expresses a β₅ integrin.

27. The method of claim 25 further comprising administering a therapy used for the treatment of luminal A subtype breast cancer.

28. The method of claim 25, wherein the β₅ integrin antagonist is selected from the group consisting of an antibody or antigen-binding fragment of an antibody, a small interfering ribonucleic acid (siRNA), a peptide, a peptidemimetic, an antisense oligonucleotide, and a small molecule.

29. The method of claim 25, wherein said breast cancer is a type selected from the group consisting of ductal, lobular and nipple cancer.

30. The method of claim 25, wherein said breast cancer tumor is a carcinoma.

31. A composition comprising a HER-2 antagonist, a β₅ integrin antagonist and a physiologically acceptable carrier.

32-33. (canceled)

34. A method for identifying a candidate for an anti-cancer therapy using a β₅ integrin antagonist comprising:
a) providing a tumor sample obtained from a subject; and
b) assessing expression of a β₅ integrin in said tumor sample,
wherein expression of β₅ integrin by said tumor or increased expression of β₅ integrin by said tumor relative to a suitable control, indicates that the subject is a candidate for an anti-cancer therapy using a β₅ integrin antagonist.

35. The method of claim 7, wherein a synergistically effective amount of said β5 integrin antagonist and said HER-2 antagonist is administered.

36-45. (canceled)
